(19) United States
(12) Patent Application Publication
(10) Pub. No.: US 2026/0297507 A1
(43) Pub. Date: Oct. 1, 2026

(54) COMPOSITION

(71) Applicant: Molecular Devices (UK) Limited, Wokingham (GB)

(72) Inventors: Jessica Pinheiro De Lucena-Thomas, Wokingham (GB); Kimberley Luetchford, Wokingham (GB)

(21) Appl. No.: 19/482,229

(22) PCT Filed: May 8, 2024
(86) PCT No.: PCT/GB2024/051190

§ 371 Date: Nov. 6, 2025

(30) Foreign Application Priority Data
May 9, 2023 (GB) 2306769.7

(51) Int. Cl. C12N 5/00

(52) U.S. Cl. CPC C12N 5/0012; C12N 5/0062; C12N 5/0068; C12N 2513/00; C12N 2533/52; C12N 2533/54; C12N 2533/74; C12N 2533/90

(57) ABSTRACT

The present disclosure provides a method of encapsulating organoids, cells or spheroids, the method comprising: (i) Mixing solutions of alginate and gellan together with buffer solution to form a mixed solution; (ii) Sterile filtering the mixed solution of (i) to form a sterile filtered solution; (iii) Suspending the organoids, cells or spheroids in a protein matrix comprising extracellular matrix protein and mixing 1:1 with the sterile filtered solution of (ii) to form a liquid suspension; (iv) exposing the liquid suspension of (iii) to calcium ions to induce gelation. A kit comprising alginate, gellan gum, and a protein matrix; and a gel composition suitable for encapsulating organoids, cells or spheroids, the gel composition comprising about 0.1% to about 1% w/v alginate and about 0.001 to about 0.02% w/v gellan gum are also provided.

### CROSS-REFERENCE TO RELATED APPLICATION(S)

This application is that national phase of PCT Application No. PCT/GB2024/051190 filed on May 8, 2024, which in turn claims priority to British Application No. 2306769.7 filed on May 9, 2023, the contents of which are incorporated herein by reference in their entireties.

### STATEMENT REGARDING FEDERALLY SPONSORED RESEARCH OR DEVELOPMENT

The project leading to this application has received funding from the European Union's Horizon 2020 research and innovation programme under the Marie Skłodowska-Curie grant agreement No. 665992.

### FIELD

The present disclosure relates to compositions for encapsulating cells and three-dimensional cell cultures, such as organoids and/or spheroids. Kits comprising the components of the compositions and methods of encapsulating cells, organoids or spheroids in the compositions are also provided.

### BACKGROUND

Three-dimensional cell cultures such organoids or spheroids typically require an extracellular matrix support to mimic the extracellular environment and act as a scaffold. Matrigel® is a gelatinous protein mixture secreted by Engelbreth-Holm-Swarm (EHS) mouse sarcoma cells that is commonly used as an extracellular matrix. The main components of Matrigel® are structural proteins such as laminin, collagen and entactin, which present cultured cells with the adhesive peptide sequences that they would encounter in their natural environment. Also present are growth factors like TGF-beta and EGF that prevent differentiation and promote proliferation of many cell types. A growth-factor-reduced Matrigel® is also available.

Organoids are traditionally encapsulated into 10-50 μm adherent droplets of Matrigel® and cultured under static conditions in well-plates or dishes with nutrients given in semi-batch (Fatehullah et al., 2016). Such Matrigel® based cell cultures have been shown to work well. However, Matrigel® is expensive and the process is labour intensive, producing small output with high levels of variability between batches. This is less problematic when cells are being cultured on a laboratory scale but means that it is not well suited to large scale production. Additionally, due to the animal derived nature of Matrigel® its composition is complex and not well defined, with a high level of variability between batches. Again, this is not suited to large scale production of organoids or spheroids as a high level of consistency is needed, especially if those organoids or spheroids are to be used for drug testing.

Synthetic alternatives to Matrigel®, such as QGel have been developed. However, the disadvantages of these alternatives include the difficulty in adapting pre-established Matrigel® cultures to an entirely new matrix, and the ability to manipulate the matrices into alternative shapes/forms is limited, especially for rapid production of large matrix volumes.

PEG-based synthetic matrices demonstrate relatively low organoid formation efficiency compared to Matrigel®. Synthetic scaffolds are often tailored to a particular cell/organoid type, whereas Matrigel® is used universally. Different organoids, as well as different cells within organoids, often require distinct physical and biochemical parameters to guide cellular behaviour, so screening multiple synthetic scaffolds can be time-consuming, cost-prohibitive and challenging.

### SUMMARY

The present inventors have recognised that there is a need for alternative scaffolds which are suitable for producing three-dimensional cell cultures such as organoids and spheroids in larger numbers and to produce organoids and spheroids of consistent form and function. This will enable wider use of organoids and spheroids in drug testing and improve the comparability of test results.

The present disclosure relates to gel compositions for encapsulating cells and three-dimensional cell cultures, such as organoids and/or spheroids. In particular, the present disclosure provides a gel composition comprising about 0.1% to about 1% w/v alginate and about 0.001 to about 0.02% w/v gellan gum. A protein matrix can be combined with the alginate and gellan gum, which balances the amount of the protein matrix with the other ingredients and allows the final protein concentration to be controlled. This is particularly advantageous when the protein matrix is a biologically sourced protein matrix, such as Matrigel®, due to the high levels of variability between different batches.

Without being bound by theory, the present inventors believe that the composition can provide sufficient biological cues from the protein matrix, while also taking advantage of the physical properties of the other components. The composition can therefore provide a gel blend capable of rapid and easy formation of self-supporting structures of any shape, such as particles or beads. These gel structures can facilitate the adaption of any protein matrix-cultured cell or organoid lines for scale up in more complex bioreactor systems, thereby allowing the production of organoids and spheroids of consistent form and function at large scale.

Kits comprising the components of the gel compositions and methods of encapsulating cells, organoids or spheroids in the gel compositions are also provided.

### BRIEF DESCRIPTION OF THE DRAWINGS

The disclosure will now be described in detail, by way of example only, with reference to the figures.

FIG. 1 shows the results of various cell lines (MG63, C3A, Iso50) tested in blend of 2% v/v Matrigel® in 1% w/v alginate. A. Alginate/Matrigel® beads with MG63 cells (osteosarcomas) encapsulated within. B. Alginate/Matrige®l beads with C3A cells (hepatocytes) encapsulated within. C. Comparison between 100% Matrigel® and Alginate/Matrigel® cultures after 6 days of incubation.

FIG. 2 shows Iso50 organoids in a gellan/Matrigel® blend or Matrigel® alone, after 3 passages in each material, and 5 days after the most recent passage. Scale bar represents 200 μM.

FIG. 3 shows Iso50 cells cultured in a blend of 1:1 Matrigel® and 5% oxidised 2% w/v alginate after 4 passages.

FIG. 4 shows beads produced from a blend of 1:1 Matrigel® and 5% oxidised 2% w/v alginate, on day 0 (day of production) and on day 6. Scale bar represents 200 μM.

FIG. 5 shows a summary of conditions investigated for successful bead formation under electrosprayed and non-electrosprayed conditions.

FIG. 6 shows the change in diameter of the gel beads over time as the beads equilibrate in culture media after being gelled in calcium chloride solution.

FIG. 7 shows the equivalent behaviour of cells in the gellan/alginate/Matrigel® (GAM) blend compared to standard Matrigel® cultures. A: representative images of organoids cultured in Matrigel® or the GAM blend showing equivalent morphology. B: Viability of cells harvested from Matrigel® or GAM cultures. No significance observed. C: Mean diameter of organoids harvested from Matrigel® or GAM cultures. No significance observed. D: Number of organoids harvested from Matrigel® or GAM cultures. No significance observed.

FIG. 8 shows organoids harvested from Matrigel® or GAM blend which were replated in Matrigel and underwent a drug response assay. The results show the organoids from the different conditions behave equivalently.

FIG. 9 shows the Young's modulus of samples of the GAM material blend and Matrigel was approximately 6 kPa, and no statistical significance was observed in the difference between the two values. Error bars represent standard deviation, n=4.

FIG. 10 shows images of organoids in beads when a cell suspension was mixed in gellan-alginate solution then Matrigel® (A) and when a cell suspension was mixed in Matrigel® and then gellan-alginate solution (B). Scale bars: 500 um.

### DETAILED DESCRIPTION

The present disclosure provides a gel composition comprising about 0.1% to about 1% w/v alginate and about 0.001 to about 0.02% w/v gellan gum. The compositions described herein are particularly suited to encapsulating cells and/or three-dimensional cell cultures. The cells, which may be in the form of three-dimensional cell cultures, are preferably eukaryotic cells, more preferably mammalian cells. Typically, cells are obtained from a multicellular organism, such as a human, and then cultured into three-dimensional cell cultures such as organoids or spheroids prior to further. The cells may be healthy or representative of disease, such as those obtained from a heathy tissue or from a disease state, for example from a malignant tumor biopsy. In some embodiments the three-dimensional cell cultures are organoids.

The term organoid simply means resembling an organ. Organoids are typically defined by three characteristics: self-organization, multicellularity and functionality (Lancaster and Knoblich, 2014). Thus, the cells arrange themselves in vitro into the 3-dimensional (3D) organization that is characteristic for the organ in vivo, the resulting structure consists of multiple cell types found in that particular organ and the cells execute at least some of the functions that they normally carry out in that organ. For example, a prototypical organoid, the mouse intestinal organoid, grows as a single-layered epithelium organized into domains such that it resembles the in vivo intestinal crypt-villus architecture, comprising the different cell types of the intestine (enterocytes, goblet cells, Paneth cells, enteroendocrine cells and stem cells) and surrounding a cystic lumen (Sato et al., 2011).

Organoids may be grown from pluripotent stem cells (embryonic or induced) or from tissue biopsies containing adult stem cells. When grown in vitro in conditions which support stem cell maintenance, organoids resemble the organ from which they were derived by recapitulating tissue specific cell types and 3D structure, genetic and physiologically relevant functions, while also being capable of continuous expansion.

The term spheroid means a three-dimensional multicellular aggregate. Spheroids are typically formed from immortalised cell lines which have been previously propagated in 2D culture conditions. While spheroids may be observed to have differing 3D morphologies, these are not necessarily representative of the original source tissue type, and the cells comprising the spheroids are typically homogeneous and not hierarchically organised.

Gellan gum is an extracellular polysaccharide secreted by the microorganism Sphingomonas elodea (ATCC 31461) previously referred to as Pseudomonas elodea. It is available in two forms, high acyl typically containing 11-13% glyceryl groups and 4-5% acetyl groups, with the total amount of the acyl groups being in the range of 15-18% (weight percent) and low acyl typically containing less than 1% of glyceryl groups and less than 1% of acetyl groups, with the total amount of the acyl groups being below 2 wt %. The gellan gum used in the compositions described herein can be a low acyl gellan gum. Low acyl gellan gum can be prepared, for example, by the methods described in U.S. Pat. No. 8,609,377.

Gellan gum forms gels at low concentrations when hot solutions are cooled in the presence of gel-promoting cations. The state of the gellan gum as described herein will be appropriate to the state in which is it being used. For example, when in a gel composition, such as a bead encapsulating organoids, cells or spheroids, the gellan gum will be in a gel state. Alternatively, when the gellan gum is being handled prior to gelation it is not in a gel state and will typically be in a sol state.

Alginate is a naturally occurring anionic polymer typically obtained from brown seaweed, which is known for its biocompatibility and ease of gelation. Alginate is known to be a whole family of linear copolymers containing blocks of (1,4)-linked β-D-mannuronate (M) and α-L-guluronate (G) residues. The blocks are composed of consecutive G residues, consecutive M residues, and alternating M and G residues. Alginates extracted from different sources differ in M and G contents as well as the length of each block. The alginate used in the compositions described herein can have a low M/G ratio (for example, G: approximately 65-70%; M: approximately 25-35%). G blocks have a higher affinity for calcium ions than M blocks. Thus, alginates with a higher M/G ratio are capable of creating more permeable, flexible and softer alginate gel matrices, whereas a lower M/G ratio leads to stronger structures.

Alginate forms gels in the presence of divalent and/or trivalent cations. The state of the alginate as described herein will be appropriate to the state in which is it being used. For example, when in a gel composition, such as a bead encapsulating organoids, cells or spheroids, the alginate will be in a gel state. Alternatively, when the alginate is being handled prior to gelation it is not in a gel state and will typically be in a sol state.

The gel composition may comprise about 0.2% to about 0.5% w/v alginate or about 0.2 to about 0.4% w/v alginate, optionally the gel composition comprises about 0.2% to about 0.3% w/v alginate. The gel composition may comprise about 0.25% w/v alginate.

The gel composition may comprise about 0.002% to about 0.01% w/v gellan gum or about 0.003% to about 0.009% w/v gellan gum, optionally the gel composition comprises about 0.004% to about 0.006% w/v gellan gum. The gel composition may comprise about 0.005% w/v gellan gum.

The gel composition may additionally comprise a protein matrix, which provides extracellular matrix (ECM) proteins that can mimic the extracellular environment and act as a scaffold. The protein matrix typically comprises ECM proteins such as collagen I and/or collagen IV, as well as laminin. The protein matrix may additionally comprise ECM proteins such as entactin, perlecan or gelatin, or a combination thereof. The protein matrix may be an artificially sourced protein matrix or a biologically sourced protein matrix.

Biologically sourced protein matrices which may be used in the compositions described herein are typically solubilised basement preparations extracted from the Engelbreth-Holm-Swarm (EHS) mouse sarcoma. EHS mouse sarcoma is a tumour rich in extracellular matrix (ECM) proteins such as laminin, collagen IV, heparan sulfate proteoglycans, entactin/nidogen, and a number of growth factors. Reduced growth factor biologically sourced protein matrices can also be used.

Suitable biologically sourced protein matrices for use in the gel compositions include protein matrices comprising laminin, entactin and collagen IV and optionally, heparin sulfate proteoglycan. Commercially available examples include Matrigel® (which comprises laminin, entactin and collagen IV), ECM Gel (which comprises laminin, collagen IV, entactin, and heparan sulfate proteoglycan), Cultrex® (which comprises laminin, entactin, collagen IV and heparin sulphate proteoglycan) and/or Geltrex™ (which comprises laminin, entactin, collagen IV and heparin sulphate proteoglycan). In some embodiments the biologically sourced protein matrix is Matrigel®.

Artificially sourced protein matrices may be PEG-based hydrogels, which are generally conjugated to key peptide residues from ECM proteins such as collagen and laminin. Polysaccharide products can also be used.

The protein matrix may form a gel in response to a chemical and/or physical signal. For example, Matrigel® forms a gel when incubated at approximately 37° C. The state of the protein matrix as described herein will be appropriate to the state in which is it being used. For example, when in a gel composition, such as a bead encapsulating organoids, cells or spheroids, the protein matrix may be in a gel state. Alternatively, when the protein matrix is being handled prior to gelation it is typically not in a gel state and may be in a sol state.

The gel composition may comprise an ECM protein content deriving from the protein matrix of about 2 mg/ml to about 10 mg/ml, or about 3 mg/ml to about 8 mg/ml. Optionally the composition comprises about 4 mg/ml to about 6 mg/ml ECM protein or about 4.25 mg/ml to about 5 mg/mL ECM protein. The gel compositions described herein therefore reduce the amount of protein matrix needed. The protein matrix component of the gel composition is therefore diluted down by the other components (i.e., the alginate and the gellan gum). Sufficient protein matrix is present to provide the biological cues needed by the cells, but problems associated with the variable nature of biologically sourced protein matrices are reduced to the dilution of this component. Dilution of the protein matrix component also allows the final protein concentration within the composition to be controlled.

The gel composition may further comprise organoids, cells, or spheroids. Optionally the gel composition further comprises organoids or cells. In some embodiments the gel composition comprises organoids.

Gel compositions disclosed herein may therefore comprise about 0.2% to about 0.5% w/v alginate, about 0.002% to about 0.01% gellan gum, about 3 mg/ml to about 8 mg/ml ECM protein and organoids, cells, or spheroids. Optionally gel compositions disclosed herein may comprise about 0.25 % w/v alginate, about 0.005% gellan gum, about 4 mg/ml to about 6 mg/ml ECM protein and organoids, cells, or spheroids.

The composition is a gel, preferably a hydrogel. Hydrogel as used herein refers to a system in which hydrophilic polymer chains are dispersed in an aqueous solution, such as an aqueous buffer solution or water. Typically, the hydrogel is in a gel state, such as a semi-solid state which retains shape. The aqueous buffer solution is optionally isotonic and/or PH neutral, both of which are beneficial for cell health. Suitable aqueous buffer solutions include phosphate buffered saline.

The gel composition typically has a Young's modulus of less than 10 kPa when measured by uniaxial unconfined oscillatory compression. For example, the gel composition may have a Young's modulus of about 5 kPa to about 10 kPa when measure by uniaxial unconfined oscillatory compression.

The present disclosure also provides a kit comprising alginate, gellan and a protein matrix. Each of the alginate, gellan and/or the protein matrix may be in the form of a solution. The alginate and the gellan may be combined. In some embodiments the protein matrix is provided separately.

The kit may include a solution comprising about 0.1% to about 1% w/v alginate and about 0.001 to about 0.02% w/v gellan gum.

The solution may comprise about 0.2% to about 0.5% w/v alginate or about 0.2 to about 0.4% w/v alginate, optionally the solution comprises about 0.2% to about 0.3% w/v alginate. The solution may comprise about 0.25% w/v alginate.

The solution may comprise about 0.002% to about 0.01% w/v gellan gum or about 0.003% to about 0.009% w/v gellan gum, optionally the solution comprises about 0.004% to about 0.006% w/v gellan gum. The solution may comprise about 0.005% w/v gellan gum.

Suitable gellan, alginate and protein matrix components for use in the kit include those described above.

The kit may additionally include instructions on how to use the kit to prepare a gel composition and encapsulate cells, organoids or spheroids using the method described below.

The present disclosure additionally provides a method of encapsulating organoids, cells or spheroids, the method comprising:
(i) mixing solutions of alginate and gellan together with buffer solution to form a mixed solution;
(ii) sterile filtering the mixed solution of (i) to form a sterile filtered solution;
(iii) suspending organoids, cells or spheroids in protein matrix and mixing 1:1 with the sterile filtered solution of (ii) to form a liquid suspension;
(iv) exposing the liquid suspension of (iii) to calcium ions to induce gelation.

The present inventors have determined that suspending cells, organoids or spheroids in the protein matrix prior to introducing them to the gellan gum/alginate mixture produces the best results. In particular, more even cell distribution has been observed, which provides more consistent conditions and ultimately a less variable end product. In contrast, mixing cells directly into the gellan gum/alginate mixture has been determined to result in poor distribution of organoids or spheroids within the gel particles or beads. Without being bound by theory, the inventors believe that calcium released from cells or present in residual traces of cell culture media causes the alginate to begin gelling around the cells, which causes the cells to form clumps.

The solutions of alginate and gellan can be mixed in step (i) at a volumetric ratio determined by the starting concentrations of the alginate and gellan gum solutions. For example, when the alginate and gellan solutions are both about 1% w/v, they may be mixed with the buffer solution at a volumetric ratio of about alginate 50:buffer 49:gellan 1.

On exposure to the calcium ions, e.g., calcium chloride ions, the liquid suspension of step (iii) changes to a gel state, i.e., to a semi-solid state which retains shape. Step (iv) may therefore comprise extruding and/or dripping the liquid suspension of step (iii) into a solution containing the calcium ions, thereby producing gel particles or beads, which contain the organoids, cells or spheroids. The shape of the gel particles or beads is not particularly limited. However, spherical or ovoid shapes might be commonly formed.

The solution containing the calcium ions is typically maintained at a temperature of about 37° C. The concentration of the calcium ions is optionally about 100 mM to about 200 mM, or about 120 mM to about 150 mM, or about 130 mM to about 140 mM. In some embodiments, the concentration of the calcium ions is about 135 mM.

The solution containing the calcium ions may additionally comprise a surfactant to decrease surface tension, which facilitates the penetration of gel droplets into the liquid and can therefore prevent the surface tension from deforming the gel particles or beads. The surfactant may be a non-ionic surfactant, preferably a polysorbate-type non-ionic surfactant. Suitable surfactants include Tween 20, Montanox 20, Polysorbate 20, PEG(20)sorbitan monolaurate, Alkest TW 20 and Scattics. In some embodiments the surfactant is Tween 20.

For example, when the liquid suspension of step (iii) is extruded from a needle, flow rate and needle size can be controlled to control the size of the gel particles or beads. Suitable needles include 25G, 26G, 27G, 28G, 29G or 30G needles, which may be used on a multi-injector manifold. Optionally the needle is 27G. The flow rate may be from about 1 mL/hr to about 20 mL/hr, optionally about 5 mL/hr to about 15 mL/hr, or from about 8 mL/hr to about 12 mL/hr. Optionally, the flow rate may be about 10 mL/hr.

The gel particles or beads may be about 200 μM to about 4000 μM in diameter. The gel particles or beads may be about 1000 μm to about 3000 μm in diameter. For example, the gel particles or beads might have a diameter of about 2.1 mm, or about 2.2 mm, or about 2.4 mm, or about 2.5 mm, or about 2.6 mm, or about 2.7 mm, or about 2.8 mm, or about 2.9 mm.

Exposure to the solution containing the calcium ions is optionally time controlled to ensure that there is no detriment to the viability or the cells, organoids or spheroids. The gel particles or beads are can be harvested immediately from the solution containing calcium ions, e.g., by using a strainer to remove the gel particles or beads. Exposure may be limited to about 10 minutes or less, or about 8 minutes or less, optionally about 6 minutes or less. The minimum exposure time may be about 30 seconds or about 1 minute.

After being harvested from the solution containing the calcium ions the gel particles or beads can then be washed, e.g., using a basal media. The gel particles or beads may then be transferred to static culture for up to 48 hours. During this time the gel particles or beads equilibrate, typically shrinking in size. For example, gel particles or beads might have a diameter of about 2.5 mm to about 2.9 mm when first formed and may equilibrate to a final diameter of about 2.3 mm to about 2.6 mm.

The liquid suspension of (iii) may comprise about 0.1% to about 1% w/v alginate, about 0.001 to about 0.02% w/v gellan gum and about 2 mg/ml to about 10 mg/ml extracellular matrix protein.

The liquid suspension of (iii) optionally comprises about 0.2% to about 0.5% w/v alginate, about 0.005% to about 0.1% w/v gellan gum and about 3 mg/ml to about 8 mg/ml extracellular matrix protein.

The buffer solution of (i) may be isotonic and/or PH neutral. In other words, the pH is typically about 7. The buffer solution does not contain any calcium as calcium ions induce gelation, which is not desirable until step (iv). Suitable buffer solutions include phosphate buffered saline (PBS).

The gel particles or beads can be dissolved in order harvest to the cells, spheroids or organoids. Optionally the gel particles or beads are dissolved using a dissolution buffer to breakdown the gel and release the cells, organoids or spheroids without damage. The dissolution buffer may comprise one or more solvents and can be optimised based on the hydrogel or hydrogels used to form the gel beads. For example, when the gel comprises a biologically sourced protein matrix such as Matrigel® the dissolution buffer preferably comprises Cell Recovery Solution, which can remove the protein matrix while avoiding dissociation of the cells of the spheroids or organoids.

Optionally the retrieved cells, organoids or spheroids may be centrifuged to separate them from any remaining culture medium after the gel has been dissolved. The centrifuge settings can be adjusted to increase the gravity and reduce the brake speed to prevent resuspension of the cell pellet.

Cell culture media are well known in the art and will be familiar to the skilled person. Typically, cell culture medium comprises amino acids, salts, glucose and vitamins and may also comprise iron and phenol red. A culture medium suitable for use in the cell expansion systems and methods described herein may be generated by modification of an existing cell culture medium. For example, the cell culture medium may be Dulbecco's modified Eagle medium (DMEM) and may comprise one or more additional components such as a nutrient mixture (e.g. Ham's F12), antibiotics/antifungals (e.g. penicillin/streptomycin), buffer (e.g. HEPES), glutamine, and n-Acetyl cysteine. The cell culture medium may additionally comprise a serum-free supplement, such as N2 Supplement and/or B27 Supplement.

### EXAMPLES

### Comparative Example 1: Matrigel® in 1% w/v Alginate

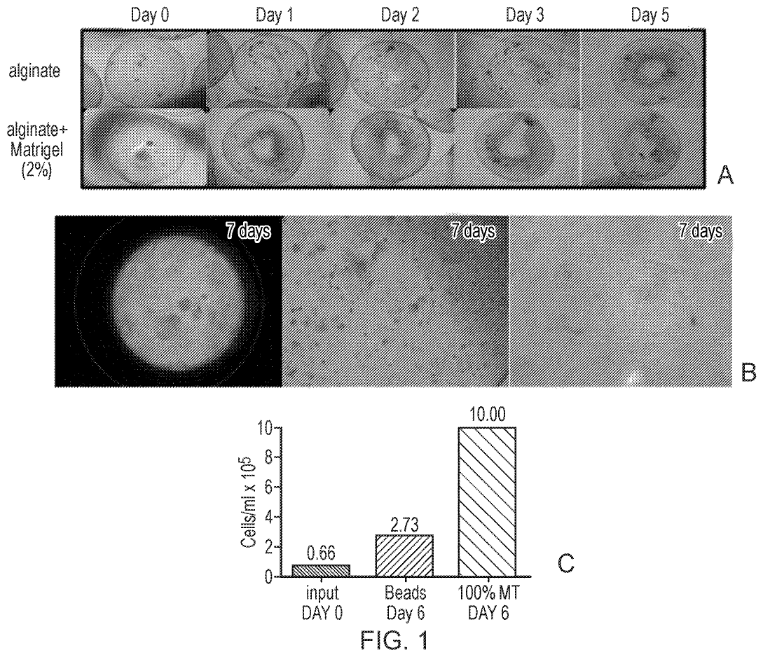

Various cell lines (MG63, C3A, Iso50) were tested in this blend. C3A mouse hepatocytes showed formation of some 3D structures (FIG. 1B). MG63 osteosarcoma cells maintained viability in the blend over 7 days (FIG. 1A). However, the only organoid line tested showed very poor growth in the blended material when compared to 100% Matrigel® cultures and this formulation was not pursued (FIG. 1C).

### Comparative Example 2: Matrigel® with Gellan Gum

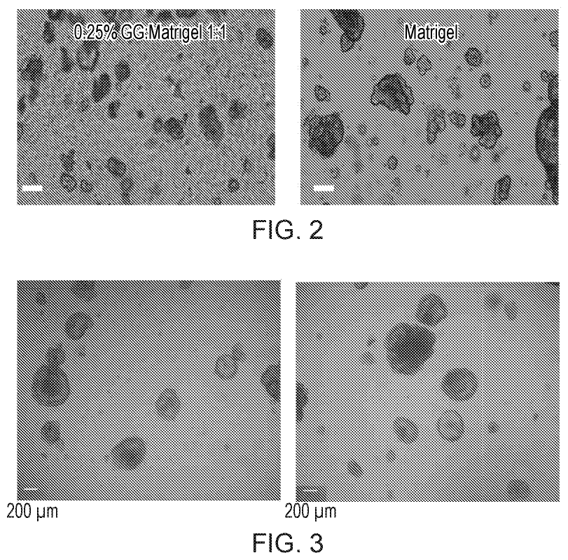

Iso50 organoids cultured in gellan gum alone at 0.25% w/v and 0.5% w/v, and in 1:1, 2:1, and 3:1 gellan gum: Matrigel® blends (over 3 passages). Only 0.25% gellan gum mixed 1:1 with Matrigel® proved successful over this time (FIG. 2). However, the gellan was difficult to work with due to its high viscosity, and it needed to be warmed up before use to decrease its viscosity in order to pipette it accurately, which conflicted with the need for Matrigel® to kept cold (~4° C.) during handling. It's likely that the relatively high stiffness of the matrices containing higher concentrations of gellan limited cell growth/organoid formation.

Bead formation was attempted 0.25% w/v gellan gum but this was not concentrated enough to form beads, so this blend was not pursued.

### Comparative Example 3: 1:1 Matrigel® with 2% w/v Oxidised Alginate

Iso50 organoids cultured over 3 passages in blends with alginate oxidised at 2.5%, 5% and 7.5% (to reduce alginate stiffness). The best results were obtained with 5% oxidised alginate in static drops/blobs of matrix (FIG. 3).

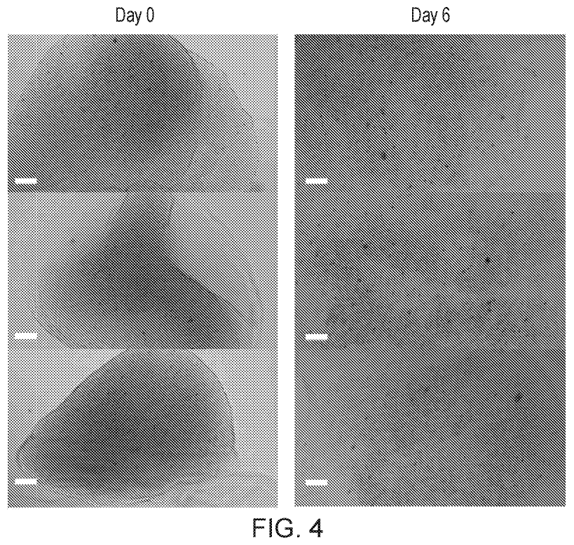

Although it was possible to form beads from this blend, the resulting structures were irregularly shaped and showed swelling/degradation over the culture period required (FIG. 4).

### Example 1: Matrigel®/Alginate/Gellan Gum

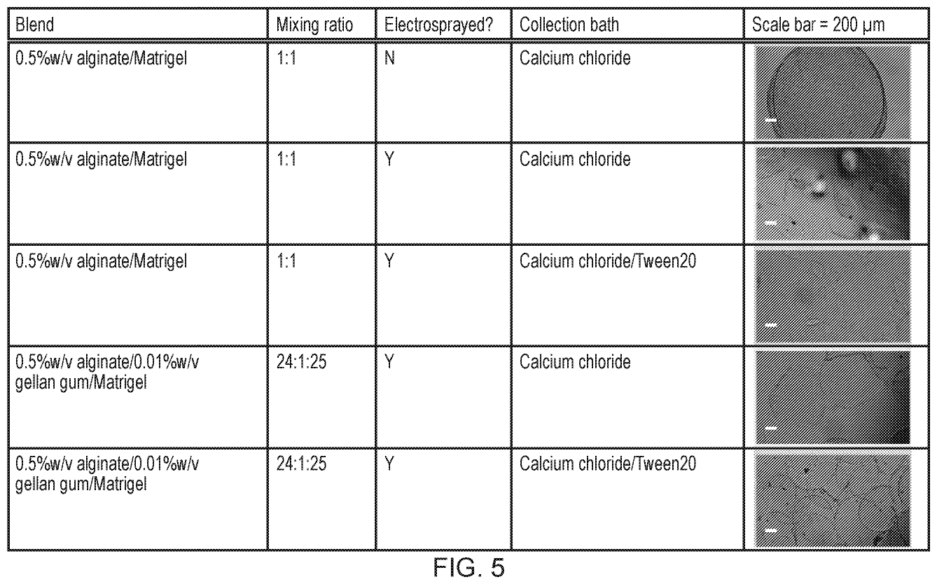

As unmodified alginate is more stable than oxidised alginate, organoid growth was tested in alginate at low concentrations (to keep matrix stiffness low). Iso50 organoids were successfully grown in blends of 0.5% w/v alginate with Matrigel® (blended 1:1). Bead formation was also shown to be possible under standard ‘dripping’ conditions (FIG. 5).

For electrospraying the blend (to produce beads of smaller diameter), the alginate/Matrigel® blend produced unevenly shaped, irregular particles. Gellan gum was introduced to the blend to increase the viscosity of the solution, resulting in more consistently shaped beads. The addition of a surfactant (Tween 20) to the gelling bath was also critical in producing round, regular beads during Electrospraying (FIG. 5).

Even when producing beads under the dripping condition (i.e., not electrosprayed) it was observed the gellan-containing matrix was more stable over time in agitated conditions.

For cell viability in the blend, the encapsulation process was optimised by limiting the length of time the matrix was exposed the gelling bath of calcium chloride/Tween 20.

### Example 2

The gel blend was produced by:
preparing a 1% w/v solution of sodium alginate in PBS;
preparing a 1% w/v solution of gellan gum in distilled water;
mixing the two together with PBS (volumetric ratio PBS:1% alginate:1% gellan gum=49:50:1);
sterile filtering; and
at the point of use, resuspending cells in Matrigel® (8.5-10 mg/mL protein concentration) and mixing 1:1 with the alginate/gellan solution.

The final concentration of the blend was Matrigel® 0.425-0.5% w/v, (4.25-5 mg/mL protein), alginate 0.25% w/v, gellan gum 0.005% w/v.

The liquid blend/cell suspension was kept on ice until use. Gelation occurred through exposure to a sterile solution of 135 mM calcium chloride (with the addition of Tween 20 for bead manufacture) for 6 minutes only.

In more detail, the GAM solution with cells was extruded at 10 mL/h through 27G needles using a syringe pump and gelation occurred by dripping the solution into a 135 mM calcium chloride bath supplemented with 1 g/L Tween 20 to decrease the bath surface tension and facilitate the penetration of gel droplets into the liquid. For processing large volumes of GAM, a 5 channel multi-injector connector with 5 attached 27G needles was used to increase the rate of bead production, and the GAM solution was extruded at 50 mL/h instead (i.e., 10 mL/h per needle). The gelation bath, held in a beaker, was heated to 37° C. to aid Matrigel® gelation, and was continuously stirred at 100 rpm using a magnetic bar and intermittently stirred with a spatula, to prevent sticking of the beads and to gently dislodge beads from the surface of the bath, respectively. After 6 minutes of bead collection, the beaker was replaced by a fresh one with the same volume of gelation bath. The beads were removed from the gelation bath by filtration and rinsed in DMEM. These 6-minute intervals were repeated as required for the volume of GAM matrix needed. The beads were combined and transferred to culture plates/dishes with basal media supplemented with 10 μM ROCK Inhibitor.

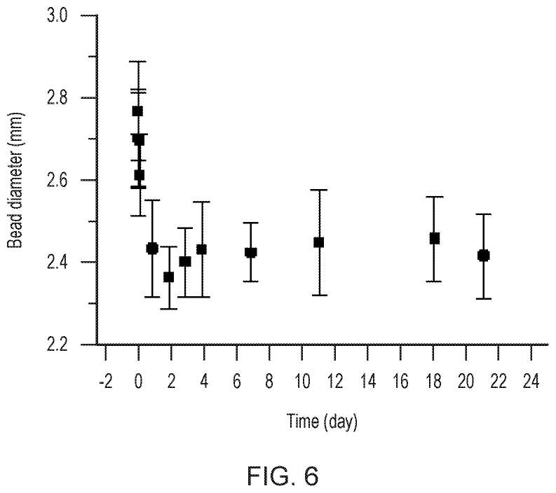

Organoids previously cultured in Matrigel® demonstrate growth in the blend consistent with their behaviour in Matrigel®. They were expanded through a modified version of the organoid expansion process (WO2018/011558) including culture under static conditions for the first 1 or 2 days. During the static culture period time, the beads shrink as shown in FIG. 6. Once they have stabilised they, are transferred into the reactor for expansion.

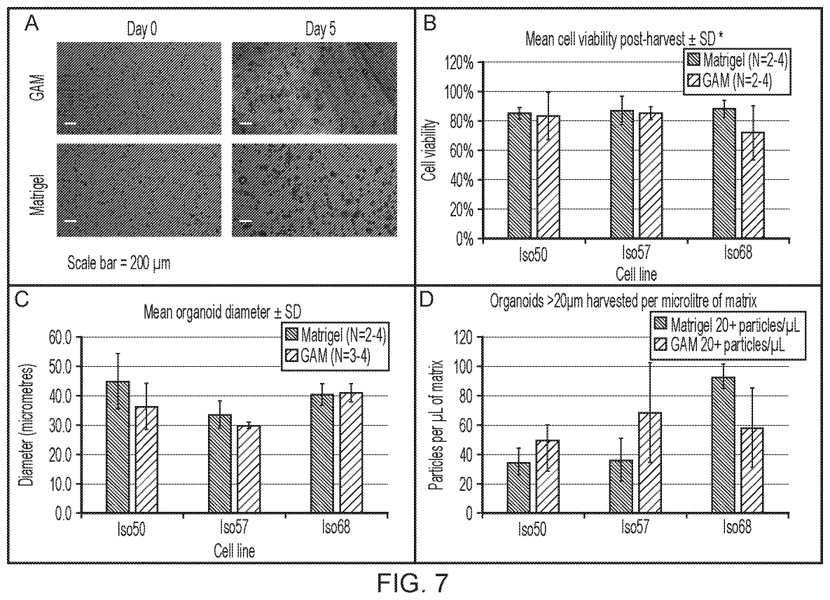

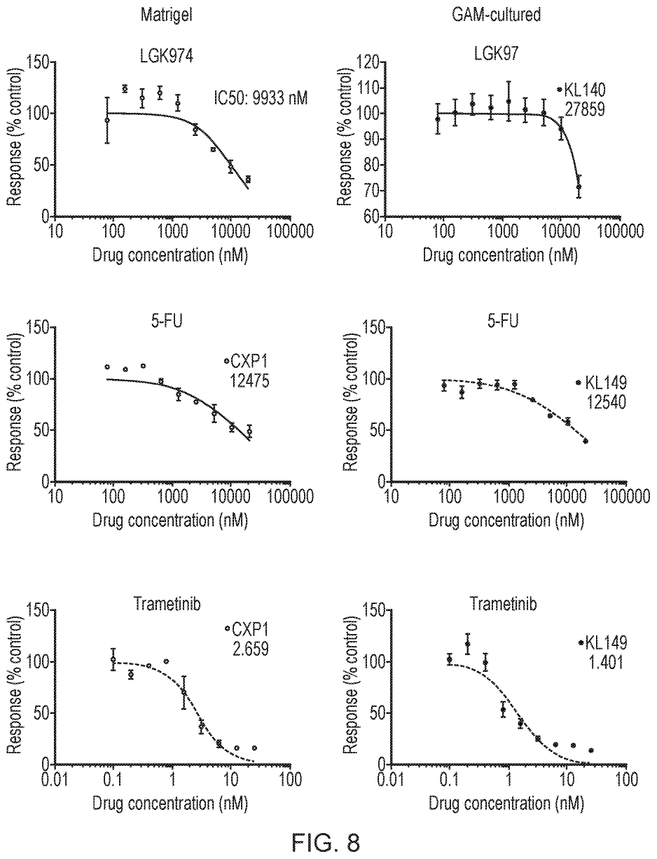

After expansion the cells were harvested by dissolving the GAM blend through physical disruption and chemical dissolution, using a solution of trisodium citrate, EDTA and sodium chloride, and a commercially available Cell Recovery Solution. The successfully harvested cells responded to a panel of drugs in a manner equivalent to the Matrigel®-only cultures. FIG. 7 illustrates the equivalent behaviour of cells in the GAM blend compared to standard Matrigel® cultures.

Once harvested from the blend culture, organoids can be replated into Matrigel® and show an equivalent functional response in example drug assays (FIG. 8).

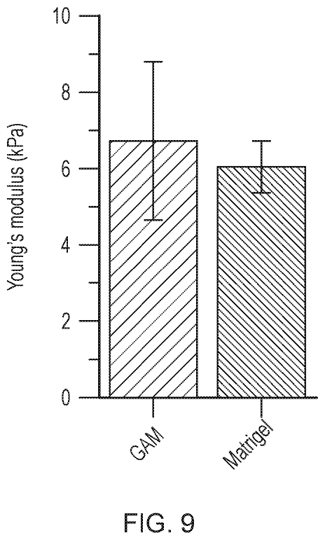

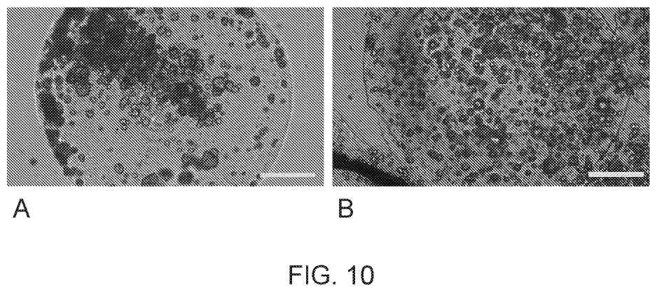

The Young's moduli of solid (gelled and hydrated) samples of the GAM material blend and Matrigel® were assessed by compression testing. FIG. 9 shows the Young's modulus of each material was approximately 6 kPa, and no statistical significance was observed in the difference between the two values.

Unlike Matrigel® alone (which gels as a result of temperature change over a period of 10-20 minutes, making it difficult to form supporting shapes during the gelling time and which is also very soft and therefore difficult to extract from a mould in an intact shape), the gel blend allows the rapid and easy formation of self-supporting structures of any shape, such as particles or beads, of solid gel matrix.

### Example 3: Blend Mixing Order

The cells, organoids or spheroids are suspended in a protein matrix comprising extracellular matrix protein, e.g., Matrigel®, and then mixed with the gellan-alginate solution, and not the other way round, to avoid premature gelation of the gellan-alginate solution when exposed to cations from the cell suspension in DMEM/F-12 cell culture media.

As shown in FIG. 9, mixing the cell suspension with Matrigel® prior to combining with the gellan and alginate solution improved cell dispersion throughout the beads (FIG. 9B) compared to cells mixed first with the gellan alginate solution and then combined with Matrigel® (FIG. 9A), especially those from large scale batches (>50 mL beads).

### References

FATEHULLAH, A., TAN, S. H. & BARKER, N. 2016. Organoids as an in vitro model of human development and disease. Nat Cell Biol, 18, 246-54.
LANCASTER, M. A. & KNOBLICH, J. A. 2014. Organogenesis in a dish: modeling development and disease using organoid technologies. Science, 345, 1247125.
SATO, T., STANGE, D. E., FERRANTE, M., VRIES, R. G., VAN ES, J. H., VAN DEN BRINK, S., VAN HOUDT, W. J., PRONK, A., VAN GORP, J., SIERSEMA, P. D. & CLEVERS, H. 2011. Long-term expansion of epithelial organoids from human colon, adenoma, adenocarcinoma, and Barrett's epithelium. Gastroenterology, 141, 1762-72.

1. A method of encapsulating organoids, cells or spheroids, the method comprising:
(i) mixing solutions of alginate and gellan together with buffer solution to form a mixed solution;
(ii) sterile filtering the mixed solution of (i) to form a sterile filtered solution;
(iii) suspending the organoids, cells or spheroids in a protein matrix comprising extracellular matrix protein and mixing 1:1 with the sterile filtered solution of (ii) to form a liquid suspension; and
(iv) exposing the liquid suspension of (iii) to calcium ions to induce gelation.

2. The method according to claim 2, wherein step (iv) comprises extruding and/or dripping the liquid suspension of step (iii) into a solution containing the calcium ions.

3. The method according to claim 1, wherein the protein matrix is an artificially sourced protein matrix or a biologically sourced protein matrix.

4. The method according to claim 3, wherein the biologically sourced protein matrix comprises laminin, entactin and collagen IV.

5. The method according to claim 4, wherein the biologically sourced protein matrix further comprises heparin sulfate proteoglycan.

6. The method according to claim 3, wherein the biologically sourced protein matrix is selected from one or more of Matrigel, ECM Gel, Cultrex or Geltrex.

7. The method according to claim 1, wherein the liquid suspension of (iii) comprises about 0.1% to about 1% w/v alginate, about 0.001 to about 0.02% w/v gellan gum and about 2 mg/ml to about 10 mg/ml extracellular matrix protein.

8. The method according to claim 1, wherein the liquid suspension of (iii) comprises about 0.2% to about 0.5% w/v alginate, about 0.002% to about 0.01% w/v gellan gum and about 3 mg/ml to about 8 mg/ml extracellular matrix protein.

9. The method according to claim 1, wherein the buffer solution of (i) is phosphate buffered saline (PBS).

10-18. (canceled)

19. A gel composition suitable for encapsulating organoids, cells or spheroids, the gel composition comprising:
about 0.1% to about 1% w/v alginate; and
about 0.001 to about 0.02% w/v gellan gum.

20. The gel composition according to claim 19, wherein the composition comprises about 0.2% to about 0.5% w/v alginate.

21. The gel composition according to claim 19, wherein the composition comprises about 0.002% to about 0.01% w/v gellan gum.

22. The gel composition according to claim 19, wherein the composition further comprises a protein matrix.

23. The gel composition according to claim 19, wherein the protein matrix is an artificially sourced protein matrix or a biologically sourced protein matrix.

24. The gel composition according to claim 23, wherein the biologically sourced protein matrix comprises laminin, entactin and collagen IV.

25. The gel composition according to claim 24, wherein the biologically sourced protein matrix further comprises heparin sulfate proteoglycan.

26. The gel composition according to claim 23, wherein the biologically sourced protein matrix is selected from one or more of Matrigel, ECM Gel, Cultrex or Geltrex.

27. The gel composition according to claim 22, wherein the composition comprises about 2 mg/ml to about 10 mg/ml extracellular matrix protein.

28. The gel composition according to claim 22, wherein the composition comprises about 3 mg/ml to about 8 mg/ml extracellular matrix protein.

29. The gel composition according to claim 19, wherein the composition further comprises organoids, cells, or spheroids.
